(19) United States
(12) Patent Application Publication
(10) Pub. No.: US 2016/0262641 A1
(43) Pub. Date: Sep. 15, 2016

(54) SENSOR APPARATUS AND METHOD FOR MONITORING A VITAL SIGN OF A SUBJECT

(71) Applicant: KONINKLIJKE PHILIPS N.V., Eindhoven (NL)

(72) Inventors: Heinz Otto KURZENBERGER, DECKENPFRONN (DE); Thomas Gerhard EMMRICH, GAERTRINGEN (DE); Bernd Günter Werner WILM, ROHRDORF (DE); Steffen ZIMMERMANN, DETTENHAUSEN (DE); Harald GREINER, NUFRINGEN (DE)

(21) Appl. No.: 15/030,407

(22) PCT Filed: Oct. 16, 2014
(86) PCT No.: PCT/IB2014/065348

§ 371 Date: Apr. 19, 2016

Related U.S. Application Data
(60) Provisional application No. 61/894,025, filed on Oct. 22, 2013.

(51) Int. Cl. A61B 5/024; A61B 5/08; A61B 5/11; A61B 5/021

(52) U.S. Cl. CPC A61B 5/02438; A61B 5/021; A61B 5/0816; A61B 5/1118

(57) ABSTRACT

A sensor apparatus for monitoring a vital sign of a subject comprises at least one sensor configured to measure a sensor signal representing a vital sign or being related to a vital sign, an evaluation unit configured to evaluate said sensor signal to determine, based on previously measured sensor signals, changes of the health condition of the subject and/or to identify an unsuccessful measurement or an unreliable sensor signal, and a control unit configured to control said at least one sensor to automatically change the measurement interval based the result of said evaluation.

### FIELD OF THE INVENTION

The present invention relates to a sensor apparatus for monitoring a vital sign of a subject. Further, the present invention relates to a patient monitoring apparatus and a patient monitoring system for monitoring vital signs of a subject. Still further, the present invention relates to corresponding methods and a computer program for implementing the method for monitoring vital signs of a subject.

### BACKGROUND OF THE INVENTION

Sensor measurements for measuring/monitoring a vital sign (e.g. heart rate, breathing (respiration) rate, SpO2, blood pressure, etc.) are conventionally either taken continuously (e.g. ECG) or aperiodic (e.g. NBP (non-invasive blood pressure)). Aperiodic measurements can be taken on demand by the user (e.g. by selecting a Start operation) or automatically, either in fixed intervals (e.g. AUTO=30 min) or in a given (i.e. preprogrammed) sequence (e.g. 4×15 min, 2×30 min, then very 60 min). The sequence allows for a more flexible scheme. It may be selected after a rather simple surgery to check vital signs first more frequent and then extend the intervals progressively as the patient is expected to recover and needs less monitoring.

Allowing a more flexible sensing sequence instead of just measuring sensor signals at fixed intervals is an improvement for most cases. However, it is known that some patients tend to e.g. slow down with their respiration rate after anesthesia and then stop breathing completely. This can be detected with a full apnea monitoring, i.e. a continuous monitoring of respiration. However, such a measurement needs to be very sensitive and therefore is prone to noise and movement artifacts so that usually a lot of false alarms are generated. In addition such measurements need to run continuously and therefore require more energy than an aperiodic measurement even if it is running more frequently.

### SUMMARY OF THE INVENTION

It is an object of the present invention to provide a sensor apparatus, patient monitoring apparatus and system as well as corresponding methods for monitoring a vital sign of a subject that are optimized with respect to energy consumption and performance of measurements.

In a first aspect of the present invention a sensor apparatus for monitoring a vital sign of a subject is presented that comprising

at least one sensor configured to measure a sensor signal representing a vital sign or being related to a vital sign,

an evaluation unit configured to evaluate said sensor signal to determine, based on previously measured sensor signals, changes of the health condition of the subject and/or to identify an unsuccessful measurement or an unreliable sensor signal, and

a control unit configured to control said at least one sensor to automatically change the measurement interval based the result of said evaluation.

In a further aspect of the present invention a patient monitoring apparatus is presented comprising

a monitor interface configured to receive sensor signals representing a vital sign or being related to a vital sign measured by at least one sensor and for transmitting control information to said at least one sensor,

an evaluation unit configured to evaluate received sensor signals to determine, based on previously received sensor signals, changes of the health condition of the subject and/or to identify an unsuccessful measurement or an unreliable sensor signal, and

a control unit configured to generate control information for control of said at least one sensor to automatically change the measurement interval based the result of said evaluation.

In a further aspect of the present invention a patient monitoring system is presented comprising

a patient monitoring apparatus as disclosed herein, and

at least one sensor configured to measure a sensor signal representing a vital sign or being related to a vital sign, said at least one sensor comprising a sensor interface for transmitting measured sensor signals to said patient monitoring apparatus and for receiving control information from said patient monitoring apparatus.

In yet further aspects of the present invention, there are provided corresponding methods, a computer program which comprises program code means for causing a computer to perform the steps of the method for monitoring vital signs disclosed herein when said computer program is carried out on a computer as well as a non-transitory computer-readable recording medium that stores therein a computer program product, which, when executed by a processor, causes the method for monitoring vital signs disclosed herein to be performed.

Preferred embodiments of the invention are defined in the dependent claims. It shall be understood that the claimed apparatus, system, methods, computer program and medium have similar and/or identical preferred embodiments as the claimed sensor apparatus and as defined in the dependent claims.

The present invention is based on the idea to make the sensor apparatus more “intelligent” and to make the measurement intervals more flexible compared to the preprogrammed measurement intervals as conventionally use. Seamless and unobtrusive measurements can thus be used in more cases where otherwise conventional continuous intensive care measurements would be indicated. The proposed (aperiodic) sensor measurements are usually non-invasive, require less energy and therefore can be done wireless with smaller batteries. They can be designed to be usually less sensitive to noise and movement artifacts. All those factors contribute to ease of use and patient comfort. The present invention thus helps to further narrow the gap that exists in monitoring performance between those measurements and the conventional continuous high end measurements still required for critically ill patients in intensive care settings.

### BRIEF DESCRIPTION OF THE DRAWINGS

These and other aspects of the invention will be apparent from and elucidated with reference to the embodiment(s) described hereinafter. In the following drawings

FIG. 1 shows an embodiment of a sensor apparatus according to the present invention,

FIG. 2 shows an embodiment of a patient monitoring system and a patient monitoring apparatus according to the present invention,

FIG. 3 shows a flow chart of a sensing method according to the present invention, and

FIG. 4 shows a flow chart of a monitoring method according to the present invention.

### DETAILED DESCRIPTION OF THE INVENTION

FIG. 1 shows an embodiment of a sensor apparatus 10 according to the present invention. It includes a sensor 12 that measures a sensor signal representing a vital sign or being related to a vital sign of a subject (e.g. a patient in a hospital, an elderly person at home or in a retirement home). Said sensor 12 may e.g. be respiration sensor for sensing the respiration rate, a blood pressure sensor for sensing the blood pressure, ECG electrodes for sensing an ECG, or a plethysmographic sensor; other examples of sensors exist. The sensor 12 is usually arranged at the patient's body, but may also be a sensor for contactless unobtrusive measurement. In other embodiments of the sensor apparatus two or more (identical or different) sensors are provided.

Further, an evaluation unit 14 is provided that evaluates the measured sensor signal to determine, based on previously measured sensor signals, changes of the health condition of the subject and/or to identify an unsuccessful measurement or an unreliable sensor signal. Said evaluation unit 14 is preferably implemented by an accordingly programmed processor or a dedicated hardware circuit. It may store previously measured sensor signals by itself, or receive those previously measured sensor signals from a memory that is internal or external to the sensor apparatus 10.

A control unit 16 is provided that controls said sensor 12 to automatically change the measurement interval based the result of evaluation performed by the evaluation unit 14. The control unit 16 is preferably implemented by a software or hardware controller or an accordingly programmed processor, e.g. the same processor that is used for the evaluation unit 14.

In some embodiments the sensor apparatus 10 is coupled to a central patient monitor (or another central unit) by a wired connection for data transfer (in particular of measured sensor signals to the central patient monitor) and for energy supply to the sensor apparatus. In other embodiments, however, the sensor apparatus 10 is coupled to a central patient monitor (or another central unit) by a wireless connection (e.g. using a WLAN, Zigbee or Bluetooth connection) for data transfer and includes its own energy supply unit 18, e.g. a rechargeable or non-rechargeable battery as indicated in FIG. 1 by dashed lines. For the (wired or wireless) connection an appropriated interface 20 is preferably provided.

The elements of the sensor apparatus 10 are preferably housed within a common housing 22, but may generally also be arranged in distributed manner and housed in different housings.

Preferably, in one advantageous implementation a method for aperiodic measurements is enabled to enter automatically a more frequent measurement mode dependent on the previously measured sensor value(s), i.e. if they indicate a worsening condition of the patient. In such a “frequent” measurement mode additional measurements are automatically taken in between the preprogrammed intervals. The extra number of measurements and/or the time between measurements can be automatically adjusted, for instance according of the prediction of the severity of the deterioration (e.g. prediction on when an alarm limit will be reached).

Thus, in an embodiment the control unit 16 is configured to control said sensor 12 to increase the measurement interval if the health condition of the subject is improving and/or to decrease the measurement interval if the health condition of the subject is worsening. Thus, the health condition of the subject directly has an influence on the automatic adjustment of the measurement interval.

In the first case (i.e. if the health status is increasing) or if the health status is stable (i.e. if the subject's health condition is neither improving nor worsening) it is preferred, not to increase the measurement interval above an upper limit set e.g. in a predetermined measurement scheme or prescribed by security rules. Thus, in case of an improvement of the subject's health condition the measurement interval is often maintained as it is. Hence, the control unit 16 is preferably configured to control the sensor 12 to measure a sensor signal at least at a minimum measurement interval.

The control unit 16 may also be configured to control the degree by which the measurement interval is changed based on the severity of the change of the health condition of the subject. Thus, if for instance the health condition is strongly decreasing the measurement interval will be reduced more strongly compared to a situation in which the health condition is only slightly decreasing, in which the measurement interval will be reduced only moderately.

Preferably, the control unit 16 is configured to control the sensor 12 to issue one of the measured sensor signals (i.e. measured within a predetermined measurement interval, e.g. within 5 min) as a kind of preliminary sensor signal, even if all those measurements were unsuccessful or all those measured sensor signals are unreliable. Measurement of the sensor signal is then repeated for an additional predetermined period (e.g. 10 min), which additional measurements may be taken “in the background” so that the user does not necessarily notice of it. The issued (preliminary) sensor signal may finally be replaced by a new sensor signal measured during said additional predetermined period, if during said additional predetermined period measurement was successful or a reliable sensor signal has been measured. Otherwise, the preliminary sensor signal is not replaced.

In another embodiment the measurement of the sensor signal may be started earlier, i.e. in advance of the planned measurement time (e.g. in a pre-measurement window) to have a sensor signal available when the measurement is due according to the planned measurement time and measurement interval.

For determining if the health condition of the subject has changed various options exists for the evaluation unit 14. In one embodiment it is determined if the measured sensor signal deviates by at least a first predetermined absolute or relative amount from one or more last measured sensor signals. In another embodiment it is determined if the measured sensor signal deviates by at least a first predetermined absolute or relative amount from an expected or estimated sensor signal trend line determined from the two or more last measured sensor signals. In still another embodiment a comparison is made of the measured sensor signal with one or more sensor signals measured by other sensors. Still further, in an embodiment information received from an external source, such as other health status sources (e.g. an early warning score from a patient monitoring apparatus) is used to determine, based on the measured sensor signal, if the health condition of the subject is improving or worsening.

In still further embodiments the evaluation unit 14 is configured to determine that the health condition of the subject has changed if a health status signal derived from said measured sensor signal deviates by at least a first predetermined absolute or relative amount from the current health status signal, from an expected or estimated health status trend line determined from the two or more last health status signals and/or from information received from another source.

In another advantageous implementation a method to retry automatically a measurement is enabled, if that measurement did not succeed, i.e. a reliable measurement could not be obtained at the automatically defined time or within a predefined time period. Hence, in such an implementation the control unit 16 is configured to control the sensor 12 to repeat measurement of the sensor signal for a predetermined period, representing a predetermined time duration or a predetermined number of times, or until the measurement is successful or the sensor signal is reliable, if an unsuccessful measurement or an unreliable sensor signal has been identified.

To determine that a measurement has been unsuccessful or that a sensor signal is unreliable said evaluation unit is preferably configured to determine if no sensor signal could be measured or if the measured sensor signal deviates by at least a second predetermined absolute or relative amount from one or more last measured sensor signals, from an expected or estimated sensor signal trend line determined from the two or more last measured sensor signals or from at least one sensor signal measured by one or more other sensors.

The first or second predetermined absolute or relative amounts and the various trend lines used in the above mentioned embodiments may be obtained empirically from earlier measurements or set by the user.

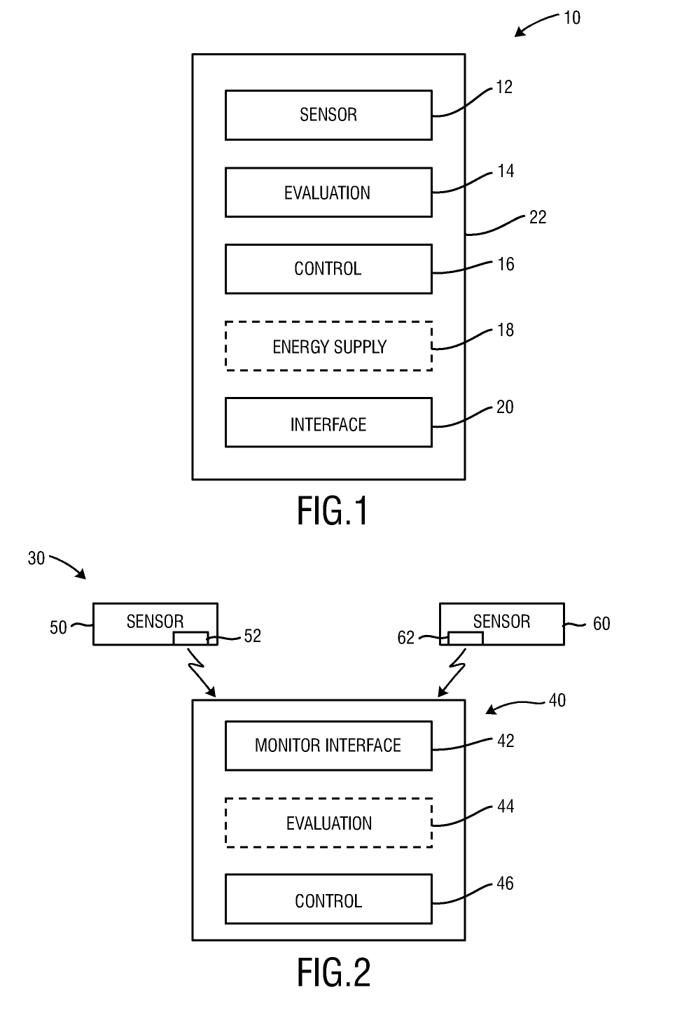

FIG. 2 shows an embodiment of a patient monitoring system 30 and a patient monitoring apparatus 40 for monitoring vital signs of a subject according to the present invention. The patient monitoring system 30 comprises said (central) patient monitoring apparatus 40 and one or more sensors 50, 60 for measuring/monitoring one or more vital signs of a subject.

The patient monitoring apparatus 40 comprises a monitor interface 42 that receives sensor signals representing a vital sign or being related to a vital sign measured by said sensors 50, 60 and transmits control information to said sensors 50, 60. An evaluation unit 44 is provided to evaluate received sensor signals to determine, based on previously received sensor signals, changes of the health condition of the subject and/or to identify an unsuccessful measurement or an unreliable sensor signal. Said evaluation unit 44 thus generally performs the same task as the evaluation unit 14 of the sensor apparatus, but performs this task for one or more sensors as a common evaluation unit. Further, a control unit 46 is provided to generate control information for control of said sensors 50, 60 to automatically change the measurement interval based the result of said evaluation. Said control unit 46 thus generally performs the same task as the control unit 16 of the sensor apparatus, but performs this task for one or more sensors as a common control unit.

While the sensors 50, 60 may generally be part of a sensor apparatus as explained above, but generally only the sensors themselves (i.e. without associated evaluation unit and control unit) are provided in said patient monitoring system 30. Said sensors 50, 60 are thus configured to measure a sensor signal representing a vital sign or being related to a vital sign. The sensors 50, 60 comprise a sensor interface 52, 62 for transmitting measured sensor signals to said patient monitoring apparatus 40 and for receiving control information from said patient monitoring apparatus 40.

It shall be noted that the patient monitoring apparatus and its elements can be further configured and has similar preferred embodiments as the sensor apparatus and as explained above.

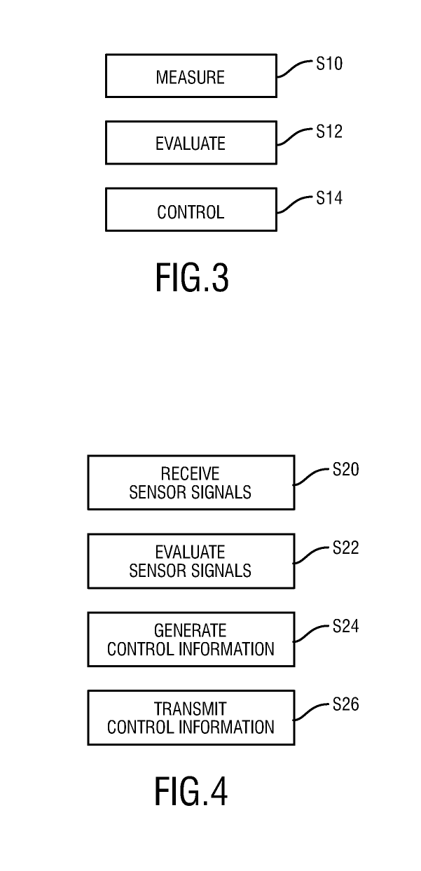

FIG. 3 shows a flow chart of a sensing method for monitoring a vital sign of a subject according to the present invention, as performed by a sensor apparatus 10 shown in FIG. 1. The first step S10 includes measuring a sensor signal representing a vital sign or being related to a vital sign. The second step S12 includes evaluating said sensor signal to determine, based on previously measured sensor signals, changes of the health condition of the subject and/or to identify an unsuccessful measurement or an unreliable sensor signal. The third step S14 includes controlling said measurement of the sensor signal to automatically change the measurement interval based the result of said evaluation.

FIG. 4 shows a flow chart of a monitoring method for monitoring vital signs of a subject according to the present invention, as performed by a patient monitoring apparatus 40 shown in FIG. 2. The first step S20 includes receiving sensor signals representing a vital sign or being related to a vital sign measured by at least one sensor. The second step S22 includes evaluating received sensor signals to determine, based on previously received sensor signals, changes of the health condition of the subject and/or to identify an unsuccessful measurement or an unreliable sensor signal. The third step S24 includes generating control information for control of said at least one sensor to automatically change the measurement interval based the result of said evaluation. The fourth step S26 includes transmitting said control information to said at least one sensor.

In summary, the general concept of the present invention applies a dynamic measurement interval depending on the patient's health condition. The trigger to change measurement interval is preferably a worsening condition of the patient.

A worsening condition can be defined if a current or averaged value (e.g. respiration rate) is above a configurable limit, representing e.g. an alarm limit of the measurement. The limit can also be dynamically adjusted depending on other patient conditions like activity or posture (e.g. if the patient is climbing up stairs and it is expected that respiration rate goes up). Therefor the limit for switching the measurement interval is adapted to be more insensitive in such a case. The evaluation unit is thus preferably configured to dynamically adjust the first and/or second predetermined absolute or relative amount depending on a physical condition of the subject, in particular to dynamically adjust the first and/or second predetermined absolute or relative amount depending on whether or not the status is performing a physical activity and/or the kind of physical activity of the subject.

A worsening condition can also be defined as change of measured value over time (trend).

The measurement interval is made longer when the patient's health condition improves to increase the sensor operating time. The measurement interval is made shorter when the patient's health condition gets worse to detect critical events earlier compared to a static measurement regime having fixed measurement intervals.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. Sensor apparatus for monitoring a vital sign of a subject, comprising:
at least one sensor configured to measure a sensor signal representing a vital sign or being related to a vital sign,
an evaluation unit configured to evaluate said sensor signal to each of: (i) determine, based on previously measured sensor signals, changes of the health condition of the subject, (ii) identify an unsuccessful measurement; and (iii) identify an unreliable sensor signal, and
a control unit configured to control said at least one sensor to automatically change the measurement interval based the result of said evaluation.

2. Sensor apparatus as claimed in claim 1, wherein said control unit is configured to control said at least one sensor to increase the measurement interval if the health condition of the subject is improving and/or to decrease the measurement interval if the health condition of the subject is worsening.

3. Sensor apparatus as claimed in claim 2, wherein said control unit is configured to control said at least one sensor to measure a sensor signal at least at a minimum measurement interval.

4. Sensor apparatus as claimed in claim 2, wherein said control unit is configured to control the degree by which the measurement interval is changed based on the severity of the change of the health condition of the subject.

5. Sensor apparatus as claimed in claim 1, wherein said control unit is configured to control said at least one sensor to repeat measurement of the sensor signal for a predetermined period, representing a predetermined time duration or a predetermined number of times, or until the measurement is successful or the sensor signal is reliable, if an unsuccessful measurement or an unreliable sensor signal has been identified.

6. Sensor apparatus as claimed in claim 5, wherein said control unit is configured to control said at least one sensor
to issue one of the measured sensor signals, even if all measurements were unsuccessful or all measured sensor signals are unreliable,
to repeat measurement of the sensor signal for an additional predetermined period, and
to replace the issued sensor signal by a new sensor signal measured during said additional predetermined period, if during said additional predetermined period measurement was successful or a reliable sensor signal has been measured.

7. (canceled)

8. Sensor apparatus as claimed in claim 1, wherein said evaluation unit is configured to determine that the health condition of the subject has changed if the measured sensor signal deviates by at least a first predetermined absolute or relative amount from one or more last measured sensor signals, from an expected or estimated sensor signal trend line determined from the two or more last measured sensor signals, from one or more sensor signal measured by other sensors and/or from information received from an external source.

9. Sensor apparatus as claimed in claim 1, wherein said evaluation unit is configured to determine that the health condition of the subject has changed if a health status signal derived from said measured sensor signal deviates by at least a first predetermined absolute or relative amount from the current health status signal, from an expected or estimated health status trend line determined from the two or more last health status signals and/or from information received from another source.

10. Sensor apparatus as claimed in claim 1, wherein said evaluation unit is configured to determine that a measurement has been unsuccessful or that a sensor signal is unreliable if no sensor signal could be measured or if the measured sensor signal deviates by at least a second predetermined absolute or relative amount from one or more last measured sensor signals, from an expected or estimated sensor signal trend line determined from the two or more last measured sensor signals or from at least one sensor signal measured by one or more other sensors.

11. Sensor apparatus as claimed in claim 8, wherein said evaluation unit is configured to dynamically adjust the first and/or second predetermined absolute or relative amount depending on a physical condition of the subject.

12. Sensor apparatus as claimed in claim 11, wherein said evaluation unit is configured to dynamically adjust the first and/or second predetermined absolute or relative amount depending on whether or not the status is performing a physical activity and/or the kind of physical activity of the subject.

13. Sensor apparatus as claimed in claim 2, wherein said control unit is configured to control said at least one sensor to start a measurement in advance of a planned measurement time.

14. Method of monitoring a vital sign of a subject, comprising:
measuring a sensor signal representing a vital sign or being related to a vital sign,
evaluating said sensor signal to each of: (i) determine, based on previously measured sensor signals, changes of the health condition of the subject; (ii) identify an unsuccessful measurement; and identify an unreliable sensor signal, and
controlling said measurement of the sensor signal to automatically change the measurement interval based the result of said evaluation.

15. Patient monitoring apparatus for monitoring vital signs of a subject, comprising
a monitor interface configured to receive sensor signals representing a vital sign or being related to a vital sign measured by at least one sensor and for transmitting control information to said at least one sensor,
an evaluation unit configured to each of: (i) evaluate received sensor signals to determine, based on previously received sensor signals, changes of the health condition of the subject; (ii) identify an unsuccessful measurement; and identify an unreliable sensor signal, and
a control unit configured to generate control information for control of said at least one sensor to automatically change the measurement interval based the result of said evaluation.

16. Method of monitoring vital signs of a subject, comprising
receiving sensor signals representing a vital sign or being related to a vital sign measured by at least one sensor,
evaluating received sensor signals to each of: (i) determine, based on previously received sensor signals, changes of the health condition of the subject; (ii) identify an unsuccessful measurement; and (iii) identify an unreliable sensor signal,
generating control information for control of said at least one sensor to automatically change the measurement interval based the result of said evaluation, and
transmitting said control information to said at least one sensor.

17. (canceled)

18. (canceled)
